(19) United States
(12) Patent Application Publication
(10) Pub. No.: US 2015/0031056 A1
(43) Pub. Date: Jan. 29, 2015

(54) DEVELOPMENT OF SPECIFIC IMMUNOASSAY CRITICAL REAGENTS FOR PHARMACOKINETIC ASSESSMENTS OF PEPTIDE POLYMERS IN PRECLINICAL AND CLINICAL MATRICES

(71) Applicants: Arumgam Murganandam, Bangalore (IN); Bindu Chikkegowda, Bangalore (IN); Konda Narasimha Venkata Raju, Perala (IN); Srikanth Sripadrao, Bangalore (IN); Santharam Muralidharan, Paramakudi (IN); Thangamma Kunjira Subramani, Virajpet (IN); Navya Shivanna, Mandya District (IN)

(72) Inventors: Arumgam Murganandam, Bangalore (IN); Bindu Chikkegowda, Bangalore (IN); Konda Narasimha Venkata Raju, Perala (IN); Srikanth Sripadrao, Bangalore (IN); Santharam Muralidharan, Paramakudi (IN); Thangamma Kunjira Subramani, Virajpet (IN); Navya Shivanna, Mandya District (IN)

(21) Appl. No.: 14/364,441

(22) PCT Filed: Dec. 12, 2012
(86) PCT No.: PCT/IB2012/057200

§ 371 Date: Jun. 11, 2014

(30) Foreign Application Priority Data
Dec. 12, 2011 (IN) 4333/CHE/2011

(51) Int. Cl. G01N 33/94; C07K 16/44

(52) U.S. Cl. CPC G01N 33/9493; C07K 16/44; C07K 2317/20
USPC 435/7.94; 436/547; 435/7.92; 436/501

(57) ABSTRACT

The present disclosure provides methods of developing a specific immunoassay for the Pharmacokinetic assessments of peptides, peptide oligomer and polymer including Glatiramer Acetate (GA), also known as Copolymer 1, Copolymer-1, Cop 1 or Cop in the clinical and preclinical matrices.

### TECHNICAL FIELD

The present disclosure provides methods of developing a specific immunoassay for the Pharmacokinetic assessments of peptides, peptide oligomer and polymer including Glatiramer Acetate (GA), also known as Copolymer 1, Copolymer-1, Cop 1 or Cop in the clinical and pre clinical matrices. The present invention also provides methods for raising pAb (Polyclonal Antibody) against Glatiramer Acetate (GA) in mammals.

### BACKGROUND AND PRIOR ART OF THE DISCLOSURE

Glatiramer acetate currently used to treat multiple sclerosis is a random Co-polymer of four amino acids found in myelin basic protein, namely glutamic acid, lysine, alanine and tyrosine. Subcutaneous administration of glatiramer acetate injection 20 mg once daily shifts the population of T cells from pro-inflammatory Th1 cells to regulatory Th2 cells that suppress the inflammatory response. Mechanism of action is attributed to its resemblance to myelin basic protein, where glatiramer act as a decoy in vivo, diverting an autoimmune response against myelin. Following SC injection of GA, it is believed that some portion of the dose may enter the lymphatic circulation and some may enter systemic circulation, however it is believed that it does not appear to cross immunopreviledged site such as the blood-brain barrier. Random polymer of GA is known to be hydrolyzed locally at injection site to small oligopeptides and free amino acids thus perpetually changing the epitopes and hence it was not possible to use Immunoassays to determine the PK profile in human beyond 1 hour after injection or in dogs 6 hours after injection (Ref. CDER Application: NDA 20-622/S015).

The new approach to raise probably high affinity pAb spanning all possible epitopes of GA, affinity purification of antibodies and isolation of GA binding serum binding proteins has allowed to develop very specific and sensitive assay to overcome the practical difficulties in detecting the Serum protein bound GA in preclinical and clinical matrices.

### SUMMARY OF THE DISCLOSURE

The present disclosure relates to a process for development of specific immunoassay for Pharmacokinetic assessments of glatiramer acetate in preclinical and clinical matrices. The said process comprises following step:
a) Method of raising pAb against Glatiramer acetate in rabbit.
b) Method of conjugating GA to BSA and affinity purification of anti-GA pAb.
c) Method of purifying the GA binding proteins from preclinical and clinical matrices.
d) Use of GA binding serum proteins or BSA or HSA as capture reagent for detection or quantification of GA in an Immunoassay.
e) Development of multiple format specific and sensitive immunoassays for quantifying GA in preclinical and clinical matrices.
f) Identification of a protein carrier to develop a novel formulation of GA for extending the half-life of the drug in vivo.

In an embodiment of the present application a cascade immunization scheme commonly used for generating antibodies against mixture of antigens was adapted to generate antibodies to the random polymer present in the GA drug substance.

In another embodiment the present invention discloses a preparation of affinity ligands such as BSA and BSA-GA conjugate for making negative and positive affinity purification columns.

In another embodiment of the present disclosure an affinity purified anti-GA Antibody specificity was assessed in an ELISA with GA, BSA-GA conjugate for positive binding and BSA, HSA, GA binding serum proteins from preclinical and clinical matrices for negative binding.

### BRIEF DESCRIPTION OF THE ACCOMPANYING FIGURES

FIG. 1 illustrates comparison of Biocon GA and commercially available GA resolved based on molecular weight in an SDS PAGE stained with silver stain (GA—Glatiramer). L1—Protein ladder, L2—commercial GA 5 μg/lane and L3—Biocon GA 5 μg/lane.

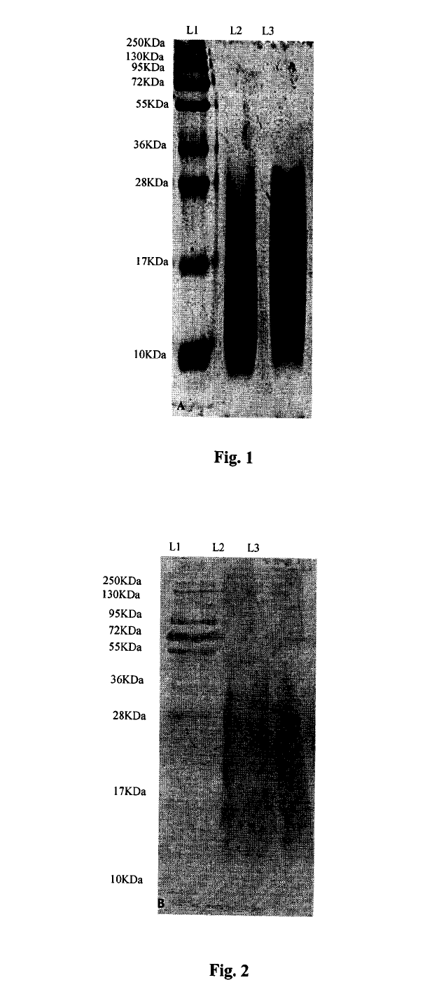

FIG. 2 illustrates identity of Biocon GA by western blotting with protein A purified Anti Biocon GA rabbit polyclonal antibodies. SDS PAGE resolved Biocon and commercial GA (5 μg) were blotted on to a PVDF membrane and probed with protein A purified Anti Biocon GA (55 μg/ml) rabbit polyclonal antibodies and detected with secondary anti rabbit polyclonal IgG ALP conjugate (dilution — 1/25000). L1—Protein ladder, L2—commercial GA 5 μg/lane and L3—Biocon GA 5 μg/lane.

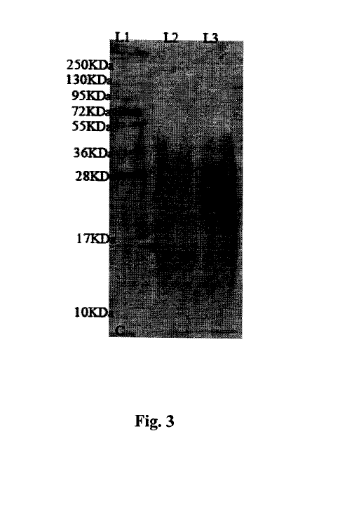

FIG. 3 illustrates identity of Biocon GA by western blotting with antigen affinity purified anti Biocon GA rabbit polyclonal antibodies. SDS PAGE resolved Biocon and commercial GA (5 μg) were blotted on to a PVDF membrane and probed with antigen affinity purified anti Biocon GA (5 μg/ml) rabbit polyclonal antibodies and detected with anti rabbit IgG ALP conjugate (dilution— 1/25000). L1—Protein ladder, L2—commercial GA 5 μg/lane and L3—Biocon GA 5 μg/lane.

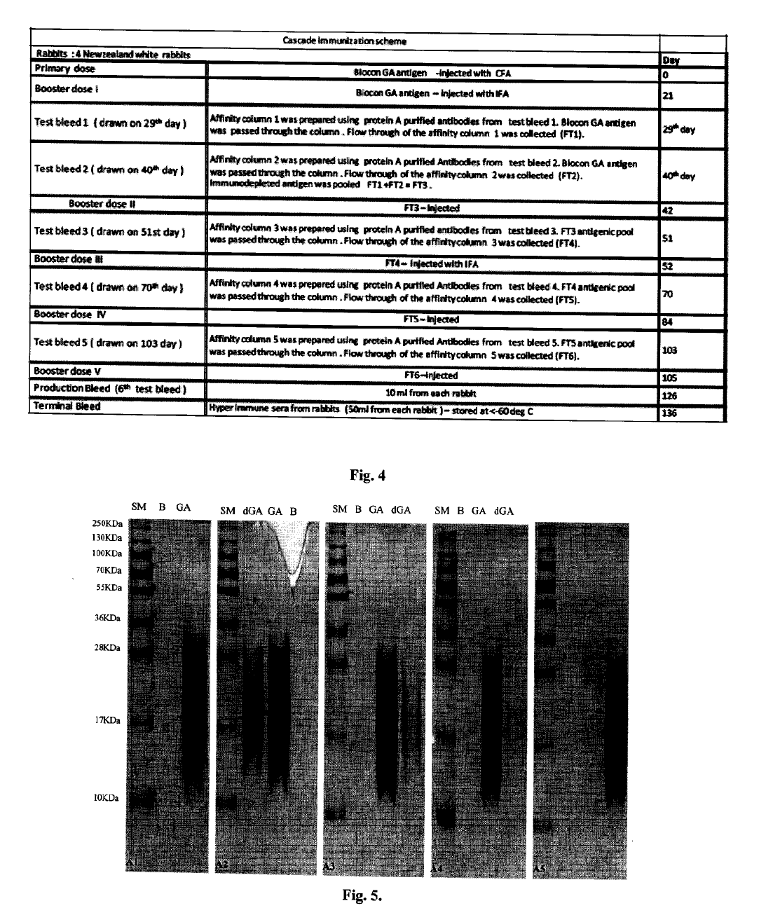

FIG. 4 illustrates cascade immunization scheme

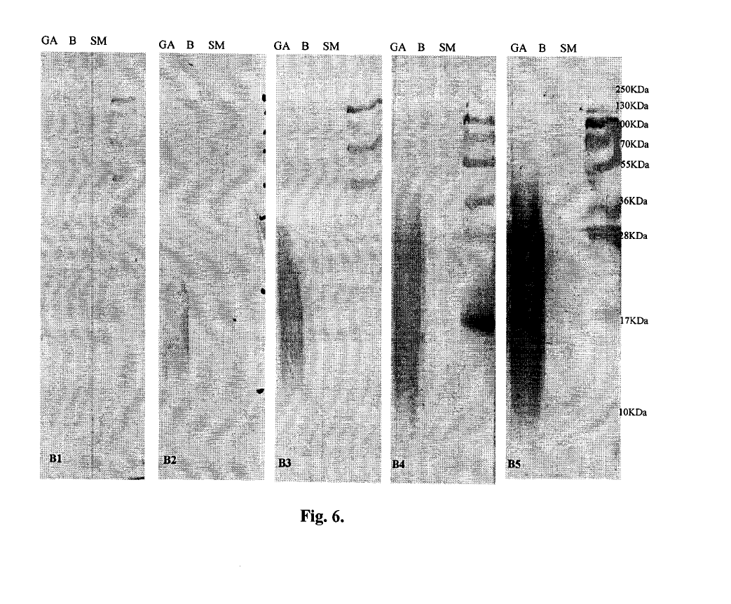

FIG. 5 illustrates SDS PAGE profiling of depleted glatiramer (GA) during cascade immunization to raise anti Glatiramer antibodies in rabbits. Lane SM: protein molecular weight marker. Lane B: Blank lane GA: Glatiramer; Lane dGA1-4; immuno depleted glatiramer antigens.

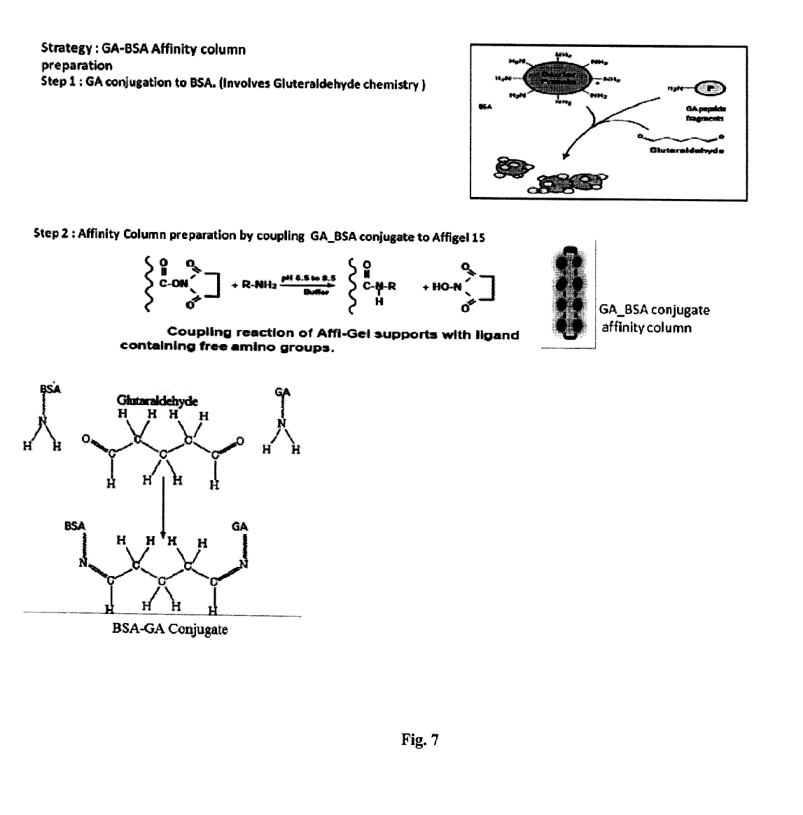

FIG. 6 illustrates Western blot analysis of glatiramer acetate with anti-glatiramer antibodies purified from pre bleed (b10 test bleed 1 (b2), Test bleed (b3), production bleed (b4) and terminal bleed (B5)

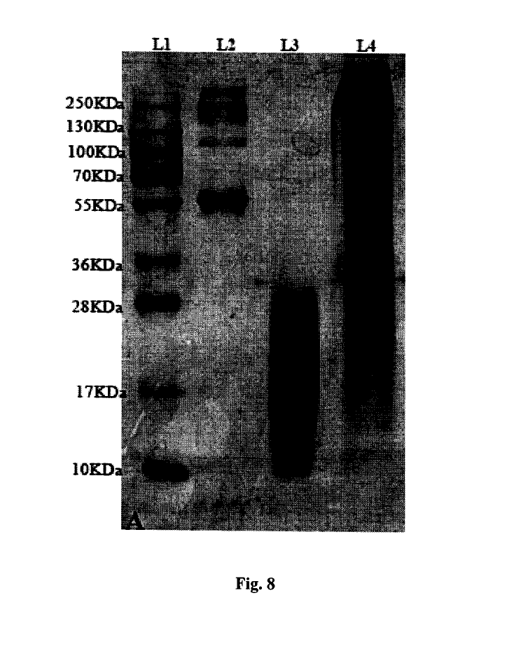

FIG. 7 illustrates GA-BSA Affinity Column preparation

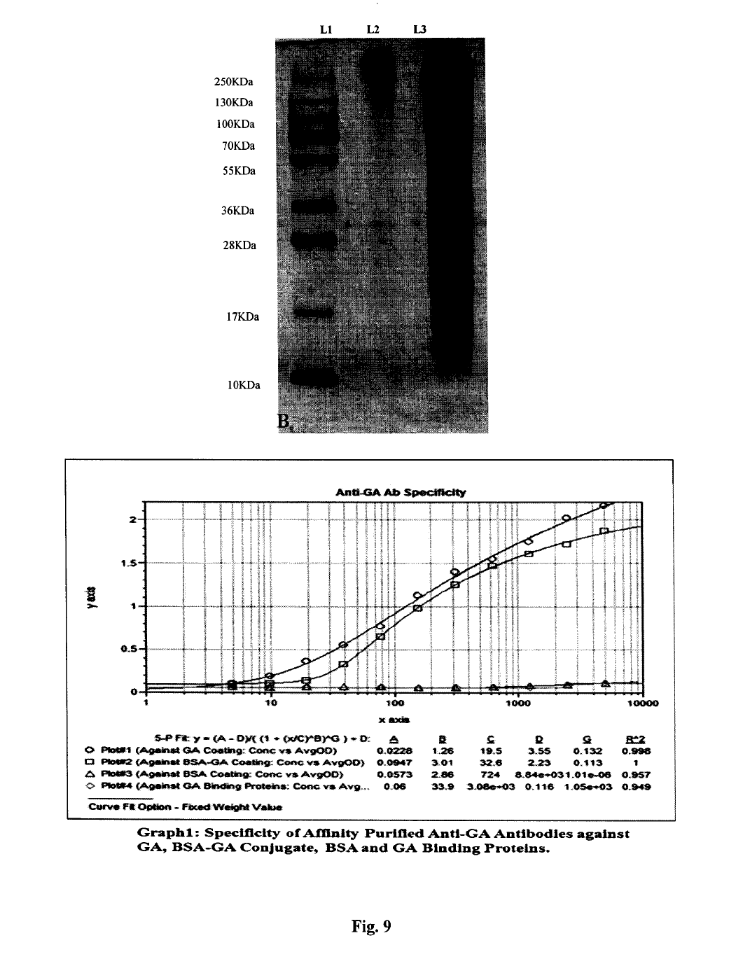

FIG. 8 illustrates cross linking of glatiramer acetate with Bovine serum albumin using glutaraldehyde as cross linker. Lane1—protein size marker, Lane 2—BSA , Lane 3—Glatiramer, Lane 4—BSA-Glatiramer conjugated (1)

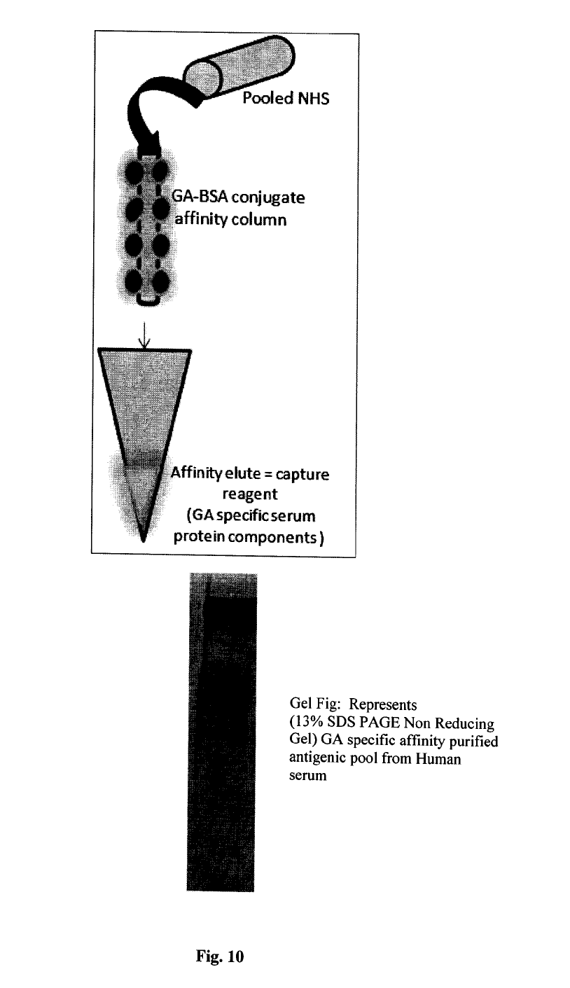

FIG. 9 illustrates efficiency of coupling of glatiramer acetate-BSA conjugate to activated sepharose matrix (affigel15) Lane 1—Protein size marker, Lane 2—After coupling (1 μg/lane), Lane 3—before coupling (1 μg/lane) (2).

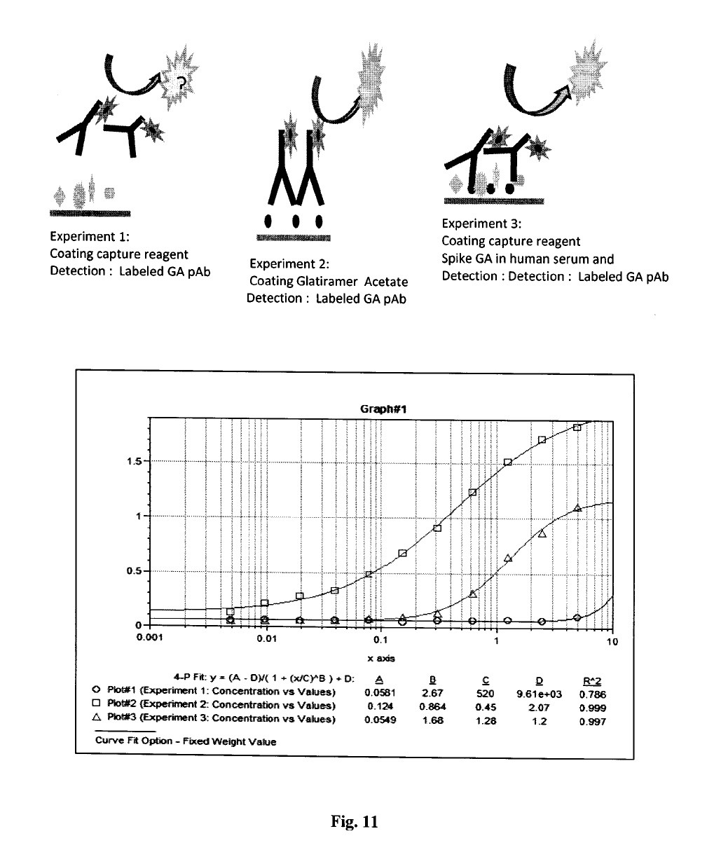

FIG. 10 illustrates purification of GA-binding proteins from matrices using BSA-GA affinity column and SDS PAGE silver staining profile of GA binding proteins from human serum.

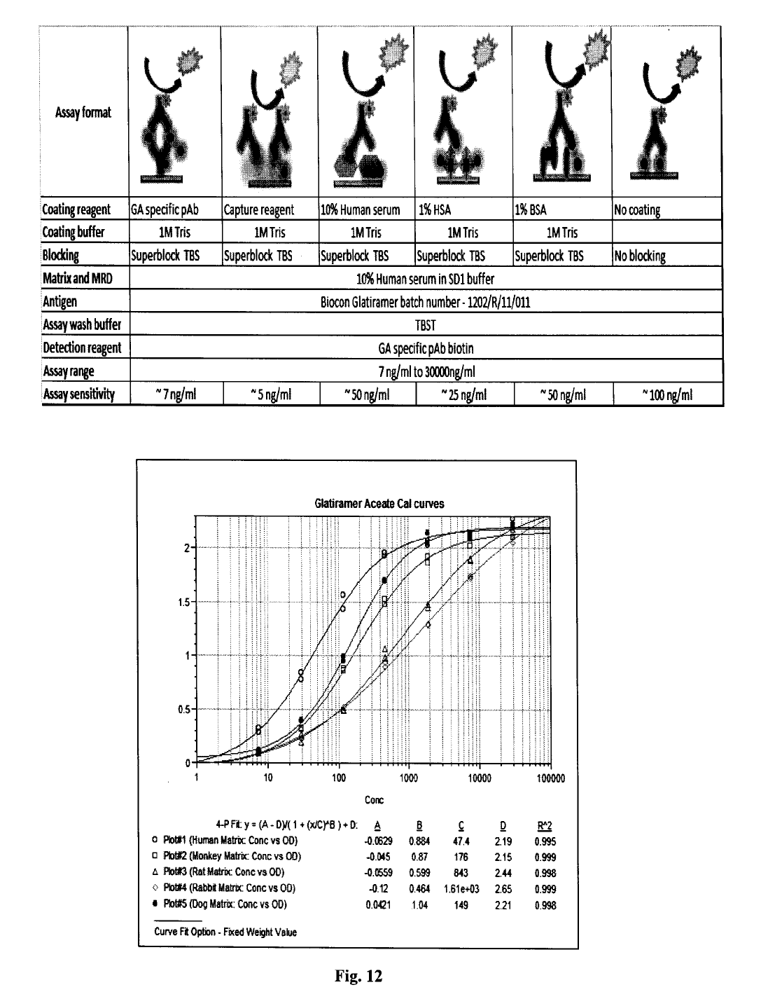

FIG. 11 illustrates specificity/cross reactivity of GA binding serum proteins

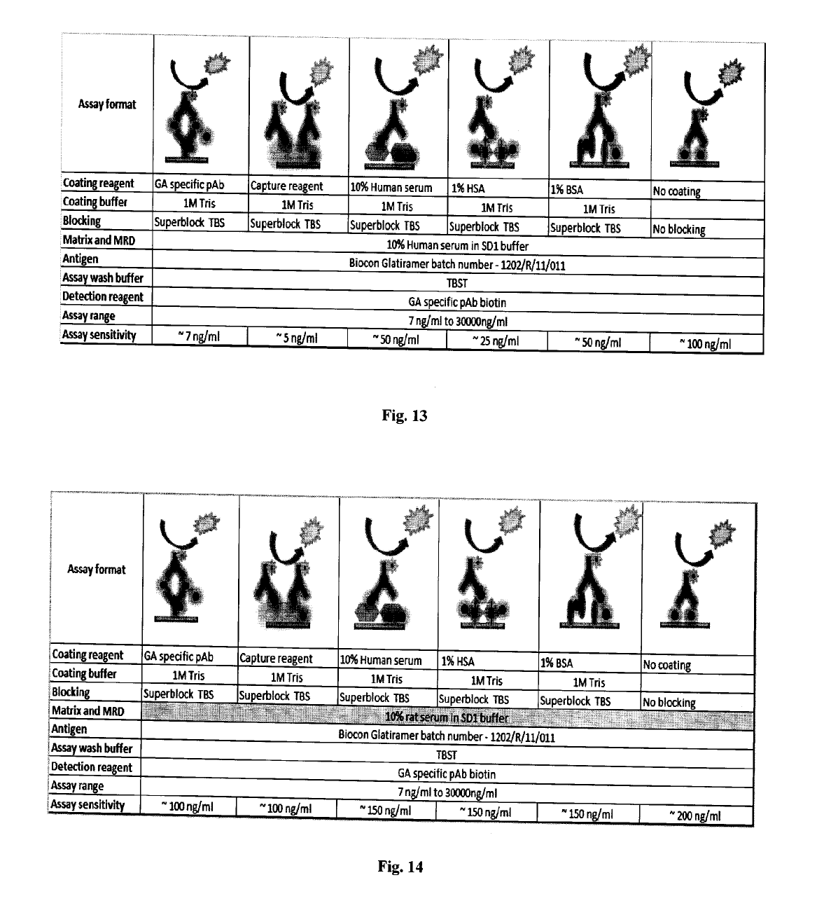

FIG. 12 illustrates immunoassay formats using GA binding serum proteins obtained from preclinical and clinical matrices as GA capture reagents.

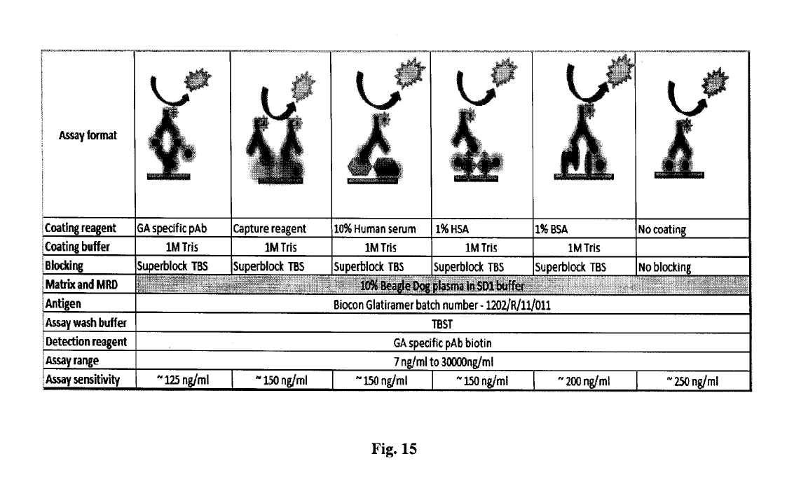

FIG. 13 illustrates pharmacokinetic assay formats developed using different GA capture reagents for detecting GA in human matrix

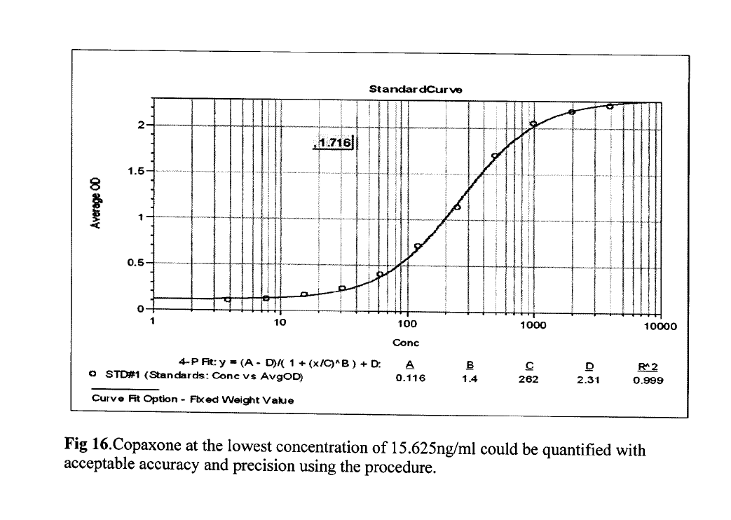

FIG. 14 illustrates pharmacokinetic assay formats developed using different GA capture reagents for detecting GA in Rat matrix

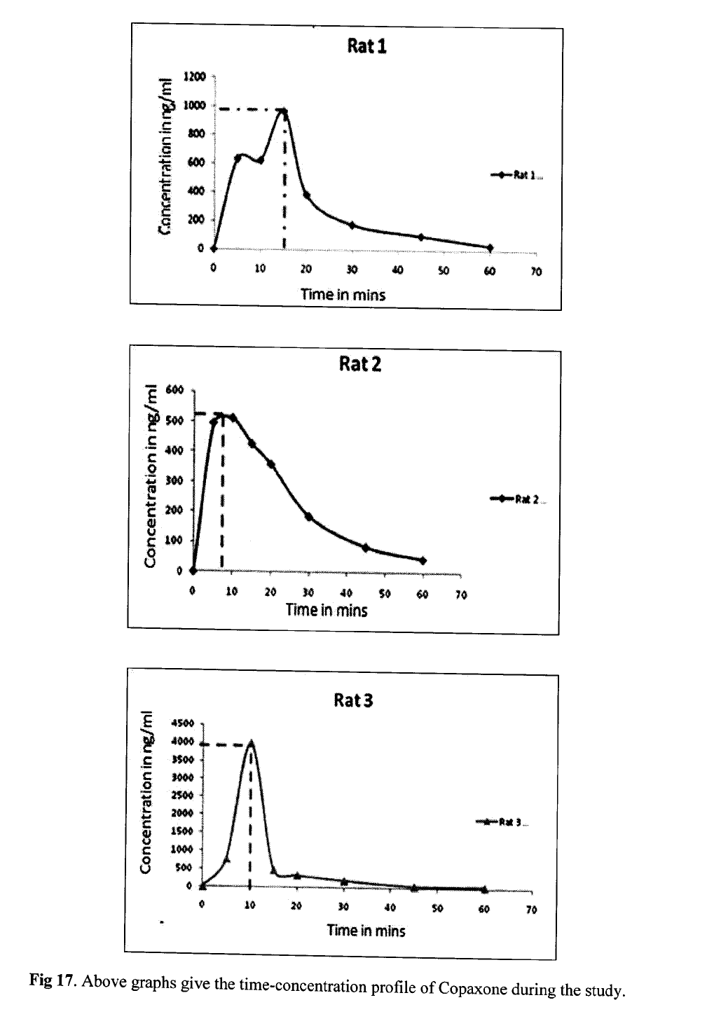

FIG. 15 illustrates pharmacokinetic assay formats developed using different GA capture reagents for detecting GA in Dog matrix

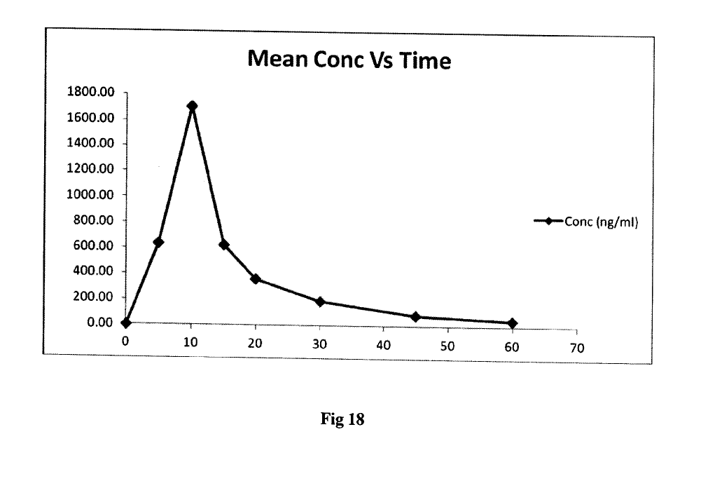

### DETAIL DESCRIPTION OF THE DISCLOSURE

The present disclosure relates to a process for development of specific immunoassay for PK assessments of glatiramer acetate in preclinical and clinical matrices. The said process comprises following step:
a) Method of raising pAb against Glatiramer acetate in rabbit.
b) Method of conjugating GA to BSA and affinity purification of anti-GA pAb.
c) Method of purifying the GA binding proteins from preclinical and clinical matrices.
d) Use of anti-GA antibodies, GA binding serum proteins or BSA or HSA as capture reagent for detection or quantification of GA in Immunoassay.
e) Development of multiple format specific and sensitive immunoassays for quantifying GA in preclinical and clinical matrices.
f) Identification of a protein carrier to develop a novel formulation of GA for extending the half-life of the drug in vivo.

In an embodiment of the present invention random polymer of GA and reference control Copaxone were resolved in SDS page gel to assess the range of molecular size of the peptides in the DP (drug product of Glatiramer acetate). The immunoreactivity of the antigens/peptides of DP were proven by a western blot technique Almost all resolved antigens/peptides of GA and Copaxone were reactive to the “protein A” purified pAb and GA affinity purified anti-GA pAb. Comparison of the data obtained from SDS PAGE and western blot profiles of GA and Copaxone suggest that by m.wt and immunoreactivity to protein A pAb and anti-GA pAb they are similar/identical.

In another embodiment a cascade immunization scheme commonly used for generating antibodies against mixture of antigens was adapted to generate antibodies to the random polymer present in the GA drug substance. In this method, after a primary and booster dose of immunization of the Rabbit with GA, the anti-GA antibodies in the hyperimmune sera of every test bleed was purified using a protein A column. Using the protein A purified Rabbit anti-GA antibodies, an affinity antibody column was prepared to selectively deplete the cognate protein antigens there by enriching for antigenic pool in the flow through of the affinity column. Since the enriched antigens failed to elicit an immune response in the previous cycle, it was used as an immunogen in the subsequent booster injection. Depleted immunoreactive antigens and enriched poorly immunoreactive antigens present in the flow through will help to prime the immune cells to generate primary (for poorly immunoreactive proteins) as well as secondary (affinity matured Ab to immunoreactive) antibodies. By repeating the cycle of purification of anti-GA antibodies, depletion of immune reactive antigens, enrichment of poorly immune reactive antigens and booster injection, we have generated antibodies to all possible immunogenic/antigenic protein populations in GA drug substance.

In yet another embodiment of the present disclosure preparation of affinity columns (BSA and BSA-GA conjugate columns) comprises the following steps:
a) BSA affinity column is prepared using Affigel 15. Coupling of BSA to affigel is carried out at pH 7.4 in PBS.
b) BSA and GA were conjugated to each other by using Glutaraldehyde chemistry linking covalently to the amine groups at N-Terminal in the respective proteins
c) Rabbit antibodies from anti-GA hyper immune serum was purified using Protein A column and the elute obtained after dialysis was passed through BSA-GA affinity column and affinity eluted the antibodies using 0.25 M Glycine (pH 2.5) and eluate were neutralized with 1 M Tris pH 9 and dialysed against PBS pH 7.4. A negative purification was performed using BSA column during one of the purification stages.

The glatiramer acetate and BSA were treated with glutaraldehyde until the cross linking reaction is completed and to check the efficiency of GA-BSA conjugates, the reaction mixture was dialyzed to remove excess unreacted glutaraldehyde and subjected to SDS PAGE followed by silver staining.

In still another embodiment of the present disclosure affinity purified anti-GA Ab specificity was assessed in an ELISA with GA, BSA-GA conjugate for positive binding and BSA, HSA, GA binding serum proteins from preclinical and clinical matrices for negative binding. Similarly, biotinylated affinity purified anti-GA Ab also demonstrated the same specificity result

The present disclosure is further elaborated by way of the following examples and accompanying figures herein. However, these examples should not be construed to limit the scope of the present disclosure.

### EXAMPLES

### Example 1

### Purification of GA Serum Binding Proteins from Preclinical and Clinical Serum Matrices

Different serum (Human, Monkey, Rat, Dog, Rabbit) was separately passed through GA-BSA conjugate affinity column, and after washing unbound proteins from the column, GA bound serum proteins were eluted using 0.25 M Glycine (pH 2.5) and eluate were neutralized with 1 M Tris pH 9 and dialysed against PBS pH 7.4. and Gel represents the strategy used to purify the GA binding proteins from matrix and the SDS PAGE profile of the same.

### Example 2

a) Specificity/Cross reactivity of GA binding serum proteins: The purified GA binding proteins were coated on to an ELISA plate and checked for cross reactivity with biotinylated GA affinity pAb along with GA direct coating as control (Experiment 2). The results demonstrated that the purified pool of antigens had no cross reactivity to affinity purified anti-GA pAb.

b) Directly coated GA and purified GA binding protein as capture reagent and detection of GA with biotinylated GA affinity pAb, gave a significant signal to noise ratio (Experiment 3). Graphical representation of the gradation observed with GA calibration curve allowed us to develop Pharmacokinetic method for detection and quantification of GA in serum Matrices.

### Example 3

### Development of PK ELISA Methods Using Purified Specific GA Binding Serum Antigens to Detect GA in Preclinical and Clinical Matrices

Purification of GA specific antigenic pool from Human, monkey, rat, rabbit and dog serum were done as described earlier and used as coating reagent for PK method development in respective matrices. A calibrator curve from 30000 ng/ml to 7 ng/mL was prepared in respective matrices and added to respective coating antigens and incubated at room temperature for 1 hour, at 5 fold MRD. Drug captured by the antigens were detected by biotinylated affinity purified Anti GA pAb followed by streptavidin-HRP conjugate. The results (Graph) demonstrated a clear signal to noise ratio in each matrix with sensitivity varying for each of the matrix (200 to 7 ng/ml), an approximate 7 ng/mL sensitivity was achieved in neat human matrix.

### Example 4

### Ranking of PK Assay Formats for Potential Use

A total of seven different ELISA methods using different immobilization reagent to capture the GA bound to serum protein have been tested using human, dog and rat matrix. Based on the preliminary results, the sandwich ELISA using affinity purified anti GA antibodies and affinity purified GA binding proteins are ideal for developing a much more sensitive and robust assays. We believe GA specific antigen and antibodies with higher affinity to GA as capture reagent in an ELISA assay will favor the dissociation (solid phase extraction) of GA bound to serum protein in samples (solution phase) and allow us to determine low concentration of GA in preclinical and clinical serum matrices.

### Example 5

### Detection of Copaxone in Serum of Sprague Dawley Rats Using Rabbit Polyclonal Anti Copaxone Antibodies

A series of serum samples, each containing Copaxone spiked at known concentration and forming a set of calibrator standards to generate a calibration curve to quantify Copaxone in the serum samples of the given species was made in the serum with Copaxone at levels 1000000 ng/ml, 30000 ng/ml, 4000 ng/ml, 2000 ng/ml, 1000 ng/ml, 500 ng/ml, 250 ng/ml, 125 ng/ml, 62.5 ng/ml, 31.3 ng/ml, 7.8 ng/ml and 3.9 ng/ml in neat serum.

ELISA plate is precoated with Polyclonal Rabbit Anti Copaxone antibodies and the appropriately diluted serum samples constituting the calibrator standards and samples for analysis were incubated for binding to the coated Anti Copaxone antibodies and unbound and poorly bound serum components were separated during washing with PBST (Phosphate buffered saline with 0.1% tween 20).The bound Copaxone was detected using biotin labeled Polyclonal Rabbit Anti Copaxone antibodies and streptavidin Alkaline phosphatase conjugate. Substrate was added to the wells and the blue colored signal obtained at 640 nm for the test samples is compared with the calibration curve prepared from concentration-response relationship of the calibrators to determine the unknown concentration of Copaxone in the serum samples.

### Example 6

### Demonstration of Specific Binding of Rabbit Polyclonal Anti Copaxone Antibodies

To specifically detect Copaxone spiked in the serum and to avoid detection of Copaxone serum protein complexes, the polyclonal Rabbit Anti Copaxone antibody was specifically purified using activated agarose columns having immobilized BSA (Bovine serum albumin) and BSA-Copaxone.

Step 1: BSA affinity column was made using Affigel 15(Biorad). Coupling of BSA to affigel was made at pH 7.4 in PBS.

Step 2: Using Glutaraldehyde chemistry BSA and GA were chemically conjugated to each other by linking covalently to the amine groups at N-Terminal in the respective proteins

### Example 7

### Pilot PK Study in Sprague Dawley Rats

Three male Sprague Dawley rats were dosed with Copaxone at 20 mg per kg body weight with a dosing volume of 1 ml at the dose concentration of 20mg/ml per day. The dosage was selected based on minimum feasible dose (based on dose volume of 1 ml per kg) for the given strength of 20 mg/ml. Each animal was weighed and dosed with Copaxone @ 20 mg/kg B.wt. Blood samples were collected in 10 μl of Protease inhibitor from each animal at the time point 5 min, 10 min, 15 min, 20 min, 30 min, 45 min, 1, 2, 3, 4, 6, 10 and 24 hours. The blood samples were stored on wet ice for serum separation. The serum was separated and stored at −80° C. till the shipment. The samples till 24 hours were shipped in Dry Ice (−44° C.).

### Summary of PK Profile in Rat Pilot Study:

| | | | | | | AUC % |
|---|---|---|---|---|---|---|
| | | | | AUC₀₋ₜ | AUC_{0-inf} | Extrap_obs |
| | T_{1/2} | Tₘₐₓ | Cₘₐₓ | (min*ng/ | (min*ng/ | (Should be < |
| Rat | (Min) | (Min) | (ng/ml) | ml) | ml) | 5%) |
| 1 | 31.18 | 15 | 968.69 | 25491.53 | 25654.06 | 0.63 |
| 2 | 23.39 | 10 | 510.79 | 15411.63 | 15705.6 | 1.87 |
| 3 | 27.79 | 10 | 3992.88 | 34732.28 | 35161.34 | 1.22 |
| Mean | 27.45 | 11.67 | 1824.12 | 25211.81 | 25507 | 1.24 |
| SD | 3.91 | 2.89 | 1892.1 | 9663.36 | 9728.7 | 0.62 |

1. A method of producing and obtaining high affinity antibodies against glatiramer acetate (GA), said method comprising:
a. immunizing a mammal with glatiramer acetate to elicit an immune response for production of antibodies against GA,
b. collecting serum from the immunized mammal comprising the produced antibodies at predetermined time after immunization and passing the serum comprising the antibodies through a Protein A column for purifying the produced antibodies to obtain a first pool of anti-GA antibodies,
c. Preparing an anti-GA antibody column and passing GA through the anti-GA antibody column of obtain a pool of unbound GA antigen; and
d. re-immunizing the mammal with the unbound GA antigen to obtain second pool of anti-GA antibodies;
e. optionally repeating the cycle comprising step b. to step d. to obtain pool of enriched antibodies that has high affinity and specificity to glatiramer acetate.

2. The method as claimed in claim 2, wherein the antibodies are polyclonal antibodies.

3. An assay for pharmacokinetic assessment of glatiramer acetate (GA) in a biological sample, said assay comprising:
a. a method for producing and obtaining antibodies against the glatiramer acetate as claimed in claim 1 and using said antibodies for preparing an anti-GA antibody column for said pharmacokinetic assessment of the sample;
b. a method of obtaining a glatiramer acetate-bovine serum albumin (GA-BSA) conjugate affinity column and using said column for:
i. positive affinity purification of anti-GA antibodies within the sample, for said pharmacokinetic assessment; or
ii. positive affinity purification of GA serum binding proteins, for said pharmacokinetic assessment;
c. a method of using a BSA or a HSA affinity column for negative purification of anti-GA antibodies, for said pharmacokinetic assessment; or
d. a method of performing ELISA using GA, purified GA serum binding protein or anti-GA antibodies, for said pharmacokinetic assessment; or
e. any combination of methods thereof.

4. The assay as claimed in claim 3, wherein the biological sample represents preclinical or clinical matrices or a combination thereof

5. (canceled)

6. The assay as claimed in claim 3, wherein the method of obtaining a glatiramer acetate-bovine serum albumin (GA-BSA) conjugate affinity column, for purifying anti GA antibodies and GA serum binding proteins comprises acts of:
a. covalently linking the amine groups at N-terminal of BSA and GA to glutaraldehyde for obtaining said GA-BSA conjugate; and
b. coupling the GA-BSA conjugate to affigel in phosphate buffer saline at pH 7.4 to obtain said GA-BSA conjugate affinity column.

7. The assay as claimed in claim 3, wherein the ELISA is Sandwich ELISA.

8. The assay as claimed in claim 3, wherein the pharmacokinetic assessment is carried out by technique selected from a group comprising enzyme immunoassay, radioimmunoassay, Western blot analysis, enzyme linked immunoabsorbant assay (ELISA) immunoprecipitation and immunoflourescence or any combination thereof.

9. The assay as claimed in claim 3, wherein the pharmacokinetic assessment provides information on T_{1/2}, Tₘₐₓ and Cₘₐₓ of the GA in the sample.

10. The assay as claimed in claim 3, wherein the GA and reference control Copaxone were resolved in SDS page gel to assess the range of molecular size of the GA peptides in the biological sample.

11. The assay as claimed in claim 3, wherein the positive affinity purification of anti-GA antibodies within the sample is carried out by passing the sample through the GA-BSA conjugate affinity column for positive binding of the anti-GA antibodies, and eluting said bound antibodies for obtaining the purified antibodies.

12. The assay as claimed in claim 3, wherein the positive affinity purification of GA serum binding proteins within the sample is carried out by passing the sample through the GA-BSA conjugate affinity column for positive binding of the GA serum binding proteins, and eluting said bound proteins for obtaining the purified proteins.

13. The assay as claimed in claim 3, wherein the negative purification of serum is carried out by passing said serum through the BSA or HAS affinity column for negative binding of anti-GA antibodies which are obtained in the flow through.

14. The assay as claimed in claim 3, wherein each of the methods within the assay provides information on specificity, cross-reactivity and concentration of the GA within the biological sample.
